(19) United States
(12) Patent Application Publication
(10) Pub. No.: US 2016/0263397 A1
(43) Pub. Date: Sep. 15, 2016

(54) INFRARED RADIATION DEVICE

(71) Applicant: VITAL TECH, PARIS (FR)

(72) Inventors: ERIC FAUCHON, BOULOGNE BILLANCOURT (FR); ALEXANDRA GAVSEVITCH, BOULOGNE BILLANCOURT (FR)

(21) Appl. No.: 15/031,584

(22) PCT Filed: Oct. 27, 2014
(86) PCT No.: PCT/EP2014/073031

§ 371 Date: Apr. 22, 2016

(30) Foreign Application Priority Data
Oct. 25, 2013 (FR) 1360480

(51) Int. Cl. A61N 5/06; A61F 7/00

(52) U.S. Cl. CPC A61N 5/0625; A61F 7/0053; A61N 2005/0659; A61N 2005/0638; A61N 2005/0666; A61F 2007/0088

(57) ABSTRACT

The present invention relates to an infrared radiation device including a mounting (2) capable of receiving a user who is lying down (9) and a covering portion (1) capable of covering the user (9) who is lying down on the mounting (2). The covering portion (1) includes a first heating layer (5a) which is capable of emitting infrared radiation (6) in the space located between the inner surface of the first heating layer (5a) and the mounting (2). The profile of the first heating layer (5a) is a parabolic curve allowing improved distribution of the infrared radiation (6).

### TECHNICAL FIELD OF THE INVENTION

The present invention relates to the field of radiation treatment, more particularly an infrared irradiation device.

### TECHNOLOGICAL BACKGROUND OF THE INVENTION

Infratherapy or the use of infrared waves in therapy allows resonance of muscular tissue, boosting cellular functions and improving blood circulation, the cardiovascular system as well as the immune system. The radiant energy of an infrared irradiation device functions by resonance frequency on the cellular membrane and produces detoxification in depth of the upper layers of the human body subjected to various contaminants. For example, this radiant energy can have a beneficial effect on wellbeing, on health or on sporting performance of a user (9) of an infrared irradiation device.

Document US 2002/0183814 discloses an infrared irradiation device comprising a support designed to receive a user lying down and half-cylindrical parts specifically for covering the user lying down on the support. The half-cylindrical parts comprise heating plates for emission of infrared radiation in the volume between the support and the half-cylindrical parts.

This device has the disadvantage of having poorly distributed infrared radiation which can be focused on a point or an axis or a limited area of the body of the user, which overheats relative to the rest of the body of the user.

### GENERAL DESCRIPTION OF THE INVENTION

The aim of the present invention is to provide an infrared irradiation system improving the properties of infrared radiation (6) and in particular having uniform distribution of the infrared emission.

To this purpose, the invention relates to an infrared irradiation device comprising a longitudinal axis parallel to which are arranged a support capable of receiving a user and a covering part capable of covering the user, the covering part comprising at least one heating layer capable of emitting infrared radiation in at least one part of the volume located between the inner face of the heating layer and the support, the device being characterized in that the profile of the heating layer, defined as the section transversal to the longitudinal axis, follows a form of parabolic curve.

According to another particular feature, the equation of the parabolic curve is y=x²−x−1.

According to another particular feature, the covering part further comprises a reflective layer of infrared radiation.

According to another particular feature, the reflective layer follows the profile of the heating layer on the outer face of the heating layer.

According to another particular feature, the support comprises a second heating layer capable of emitting radiation in at least one part of the volume between the inner face of the first heating layer and the support.

According to another particular feature, an end, according to the longitudinal axis, of the covering part comprises a third heating layer closing the covering part.

According to another particular feature, the covering part comprises at least two portions of parabolic profiles, the second portion of the covering part being capable of sliding relative to the first portion parallel to the longitudinal axis so as to retract in or on the first portion (1a).

According to another particular feature, the first portion is secured to at least one slide capable of guiding in a slider.

According to another particular feature, the second portion is secured to at least one slider on its inner face capable of sliding on at least the slide of the first portion.

According to another particular feature, the covering part comprises an outer layer protecting the interior of the covering part.

According to another particular feature, the outer layer has a form which follows the form of the heating layer.

According to another particular feature, the heating layers comprise at least one carbon plate.

According to another particular feature, the reflective layer comprises at least one layer made of aluminium.

According to another particular feature, the outer layer is made of wood.

According to another particular feature, the support is made of wood.

According to another particular feature, the inner surface of the covering part is covered in textile.

According to another particular feature, the irradiation device comprises a lifting system of the covering part, the first portion or the second portion of the covering part being fixed on at least the lifting system of the covering part, the lifting system of the covering part comprising at least one thrust jack whereof an end is fixed on the support or on the feet of the support and the other end is fixed on the first or the second portion of the covering part such that the jack can exert thrust tending to lift an end of the covering part, the other end of the covering part being fixed on the support by means of a hinge allowing the longitudinal axis of the covering part to form a non-zero angle with the plane of the support.

The invention, with its characteristics and advantages, will emerge more clearly from the description given in reference to the appended drawings, in which:

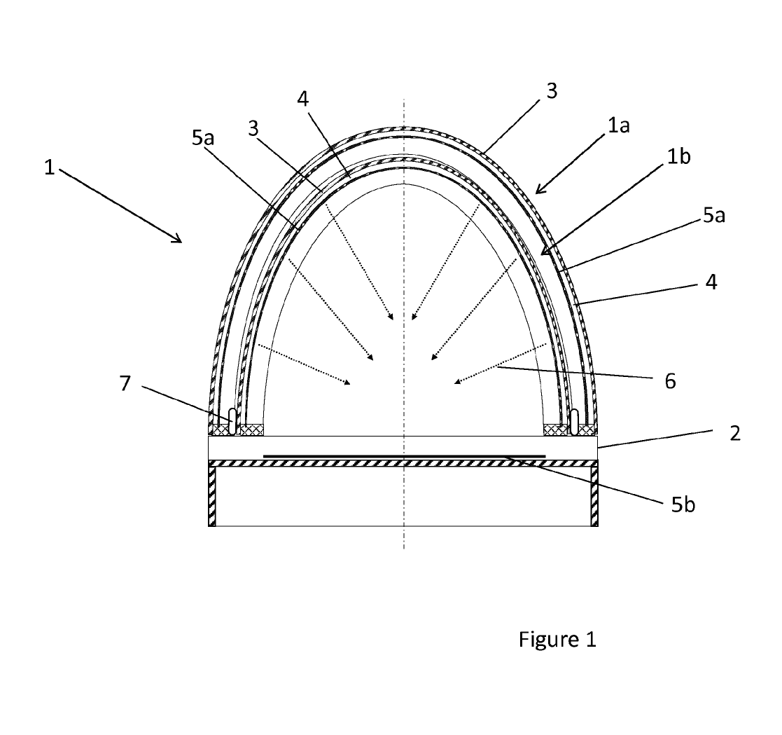

FIG. 1 illustrates a section of the device according to a plane perpendicular to the longitudinal axis of the device.

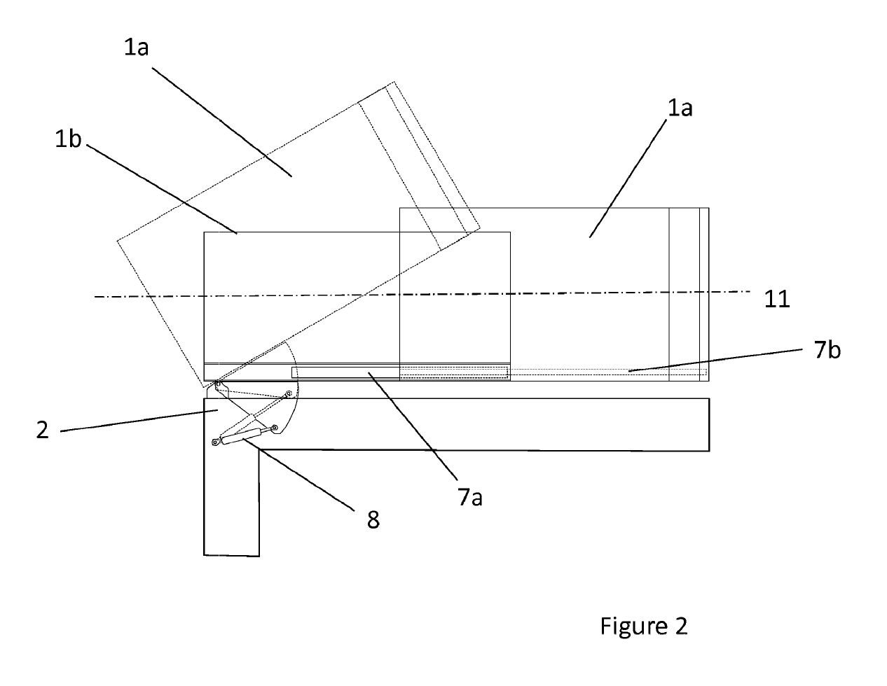

FIG. 2 illustrates a profile view of the device according to two positions of the covering part.

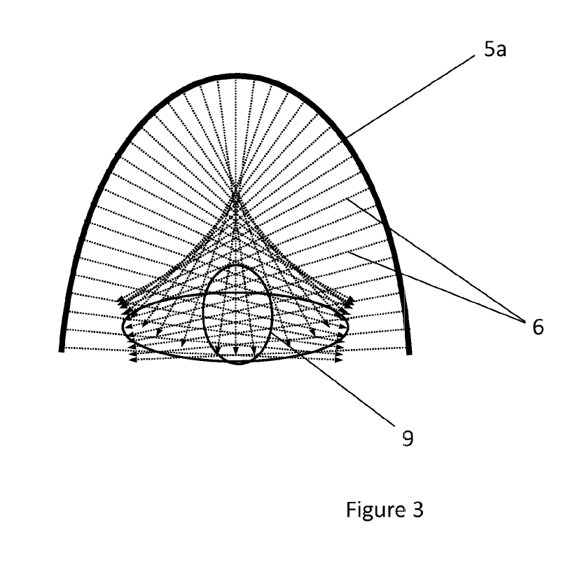

FIG. 3 illustrates the distribution of the infrared radiation in a plane perpendicular to the longitudinal axis of the device.

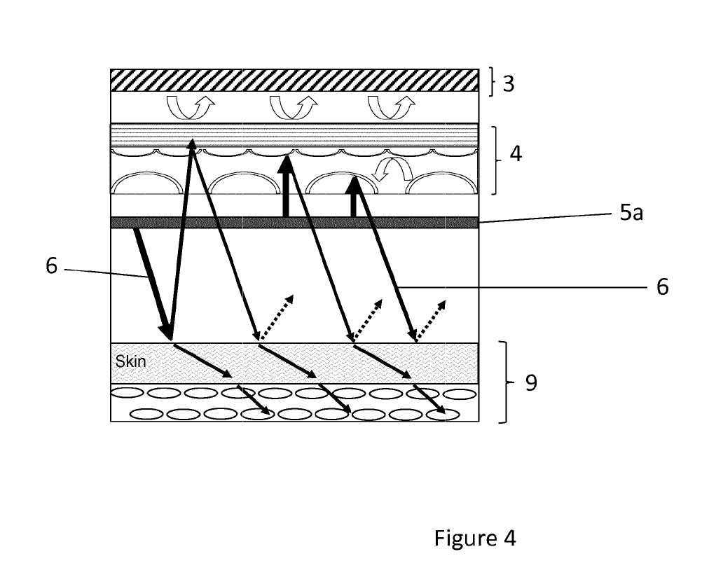

FIG. 4 illustrates schematically the performance of infrared radiation between the covering part and the user of the device.

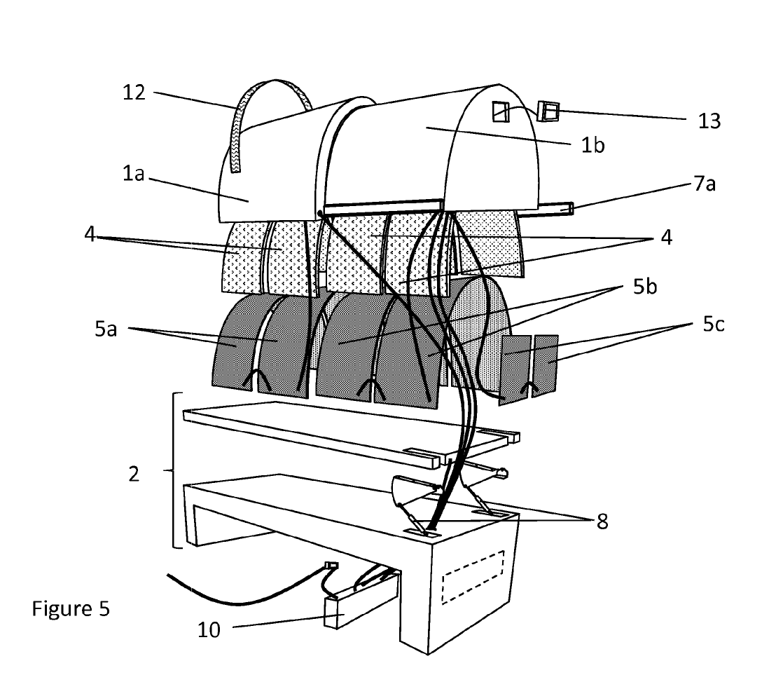

FIG. 5 illustrates an exploded view in perspective of the device.

### DESCRIPTION OF PREFERRED EMBODIMENTS OF THE INVENTION

The rest of the description will make reference to the figures cited hereinabove.

The invention relates to an infrared irradiation device. This device is sometimes also called an individual sauna. In the rest of the description, the device could be called irradiation device or simply device.

In differing from a traditional sauna which heats a body indirectly by heating ambient air, the infrared irradiation device heats the body directly by heat produced by infrared radiation (6). The temperature reached by the device can advantageously be in a range varying from ambient temperature to around 80° C., but can extend to temperatures reached by traditional saunas as high as 105° C. The ambient temperature is generally a temperature equal to 20° C. or slightly less than 20° C., but ambient temperature can go to 35° C. or 40° C.

The infrared irradiation device comprises a support (2).

This support (2) receives a user (9), for example, lying down.

The support (2) is surmounted of the covering part (1) which covers and borders the sides of the user (9) who is for example lying down on the support (2).

The support (2) and the covering part (1) are arranged according to a longitudinal axis of the device.

The covering part (1) comprises at least one heating layer.

In some embodiments, this layer comprises at least one carbon plate. In the present description the heating layers are designated therefore sometimes, non-limiting, by the term “carbon plates”. The carbon plates are generally produced from superconducting polymer tissue composed of carbon, silica and graphite. The tissue is preferably insulated between two epoxy laminated fiberglass sheets and rolled at high temperature. The strong density and regular distribution of carbon allow regular emission of the infrared radiation (6) over its entire surface. A fiberglass sheet generally comprises on two edges opposite the carbon plate, material conducting electricity such as copper in contact with the tissue and fed by electricity by means of a controller (10). For example, the two edges have two copper strips feeding the tissue.

The covering part (1) comprises at least one heating layer (5a, 5b).

This heating layer (5a, 5b) is capable of emitting infrared radiation (6) in at least one part of the volume located between the inner face of the heating layer (5a, 5b) and the support (2).

A particular feature of the infrared radiation (6) is emission of waves of the heating layer (5a, 5b, 5c) which causes a rise in temperature of the user (9) receiving the waves emitted. In a non-limiting way, the waves emitted have wavelengths comprised between 0.76 μm and 10 μm.

The advantage of this heating mode is that infrareds are not or poorly absorbed by air, while solids and liquids absorb them easily.

The wavelengths are inversely proportional to the temperature of their emitter. According to Wien's law, the higher the temperature of the emitter, the shorter the wavelength.

Absorption by the biological tissues of photons of infrared waves modifies the state of vibration or molecular rotation. Due to their low energy, infrared photons cannot produce ionisation, or photochemical reactions. Infrared radiation is overall seen as a heat source producing relaxation and wellbeing, or even added advantages as described in the present application.

In some embodiments, the heating layers of the device emit long infrared radiation (6) in the wavelength range comprised between 6 μm to 19 μm.

The human skin is complex material considered as a grey body whereof the emissivity is close to that of a black body. It dissipates its heat partly by radiation. For a black body, the Stefan-Boltzmann law integrates the notion of emissivity which illustrates a link between the energy radiated by a surface undergoing an incident flow and the energy which a black body would radiate at the same temperature. Emissivity can vary as a function of wavelength. Wien's displacement law is obtained by derivation of Planck's law. It gives the wavelength corresponding to the maximum spectral radiation of a black body as a function of its temperature. From this law, it is seen that at a temperature of 35° C. the skin emits maximum energy at a wavelength of 9.4 μm. At a temperature of 37° C., the skin emits maximum energy at a wavelength of 9.3 μm. At a temperature of 38.4° C., the skin emits maximum energy at a wavelength of 9.3 μm.

Consequently, the heating layers of the infrared irradiation device preferably emit at a length of between 8.7 μm and 9.5 μm. More preferably, they emit at a wavelength of 9.3 μm.

Also, it is known that some bodies, especially comprising carbon and silicon can emit infrared radiation which spreads in a preferred direction perpendicular to its surface, for example as is demonstrated in the doctorate thesis of Martine Laroche, (Role of surface waves in the modification of radiative properties of microstructured materials. Application to designing infrared sources and to the thermophotovoltaic effect. École centrale, Paris, 2005). So, the heating layer(s), preferably comprising carbon, silicon and graphite, utilised in some embodiments of the present application, emit infrared radiation whereof the preferred direction is perpendicular to their surface at all points. This directional radiation can be felt by the fact that the heating layer(s) heat the space facing it and not to the side. The advantage of this is to heat only the space inside the device and limit ambient heating in the room where the device is located. Also, various embodiments take advantage of this directional radiation by using a particular profile.

In fact, in some preferred embodiments, the section of the heating layer (5a, 5b) transversal to the longitudinal axis advantageously follows a form representative of a parabolic curve. Here this form of the transversal section is designated by the term profile and parabolic profile in particular. Also, as the device can comprise several heating layers, this heating layer with parabolic profile is designated here by the term “first heating layer” but this term must not be interpreted as limited as it in fact designates the radiating part of the dome above the user and is composed of one or more heating layers.

The parabolic profile of the first heating layer (5a, 5b) produces a convergence area of the infrared radiation (6) best distributed over the entire body of the user (9). For example, as shown in FIG. 3, infrared radiation is emitted according to a preferred direction perpendicular to the surface of the heating layer at each point of the heating layer. The combination of the parabolic profile with this preferred perpendicular direction enables homogeneous distribution of the radiation. In the convergence area, the temperature value generated in Kelvin can be multiplied by a factor of 1.4 for two beams which cross.

If the first heating layer (5a, 5b) had a profile of half-circular form, the infrared radiation (6) of the convergence area would reduce at a single point which would be the centre of the circle of the half-circular form. The consequence of this is poorer distribution of the infrared radiation (6) which is focused on an axis parallel to the longitudinal axis of the device, while with a parabolic profile the distribution can advantageously be adjusted to at least approximately follow the outer form of the body of the user present inside the device. In this way, apart from the comfort of the user, such heat distribution grants the device according to the invention applications in phlebology such as for example reduction in varicose veins, which would be unlikely for a device of the prior art.

In some embodiments, the function of the parabolic curve is y=x²−x−1. It is evident that the golden ratio

$$\frac{1 + \sqrt{5}}{2}$$

is the positive solution for x²−x−1=0, where x illustrates the coordinates according to the axis of the abscissae parallel to the support (2) of the device and y illustrates the coordinates according to the axis of the ordinates perpendicular to the support (2) of the device with the axis of the ordinates directed downwards.

In some embodiments, for example at the controller (10), the device comprises at least one display means for displaying the wavelength emitted by the heating layer fed by means of the controller (10). Such a display can also be produced on display means (13) of the device. In fact, in some embodiments, the device comprises display means (13) for displaying information on the operation of the device, such as the temperature produced by the heating layers, the time of use of the device or other useful parameters for operating the device.

In some embodiments, the covering part (1) of the infrared irradiation device also comprises a reflective layer (4) of infrared radiation (6) on the outer face of the heating layer. The profile of the reflective layer (4) preferably follows the profile of the heating layer (5a, 5b). This reflective layer (4) reflects infrared radiation (6) which has not been absorbed by the body of the user (9) to be sent back to the body of the user (9). This allows energy-saving necessary for emitting the infrared radiation (6). This also allows safety from the infrared irradiation device, as the outer surface of the covering part (1) stays cold.

In a configuration, the reflective layer (4) comprises at least one film or a plate made of aluminium. In addition or as an alternative, it can also comprise at least one film or a plate containing cobalt, particularly effective for reflecting infrared.

In some configurations of the reflective layer (4) it comprises several components. In a preferred configuration, the components comprise six reflective films, two layers of cotton wool and six layers of foam.

In some embodiments, the covering part (1) of the device comprises an outer layer (3) protecting the interior of the covering part (1). In some preferred embodiments, this layer (3) is rigid. In some embodiments, this outer layer (3) is made of plastic material or metal, or is made of wood. In some embodiments, the outer layer (3) can be made of fiberglass or carbon fibers or any moldable fiber. In some embodiments, the outer layer (3) can be made by a combination of materials mentioned above.

The reflective layer (4) of the device is arranged between the outer layer (3) and the heating layer. The heating layer therefore has no contact with the outer layer (3) of the device, which considerably limits energy transfers by conduction and produces good thermal efficacy relative to the prior art.

In some embodiments, the outer layer (3) has a profile of parabolic form which follows the profile of the heating layer. For example, the reflective layer (4), the outer layer (3) and the first heating layer (5a) are fixed relative to each other. They are preferably fixed by fastening means supporting heat and do not cause emission of vapors, or even insulation between at least 2 of these 3 layers, for example by way of a space (e.g., an air gap) between at least 2 of these layers, in particular between the reflective layer (4) and the outer layer (3). The fastening means are for example grooves in which are nested the ends of the different layers. These grooves are arranged for example on at least two edges located at the opposite ends of the outer layer and in which the heating and reflective layers are inserted.

In some embodiments, the support (2) comprises a second heating layer (not shown) capable of emitting radiation in at least one part of the volume between the inner face of the heating layer and the support (2). For example, the second heating layer is integrated into a mattress, for example made of damping or comfortable material, placed on the support (2).

In some embodiments, a first end of the covering part according to the longitudinal axis (11) is closed. In some of these embodiments, this first end of the covering part (1) also comprises a third heating layer (5c) which heats the region of the feet of the user.

In some embodiments, the second end is open.

In some embodiments, the second end of the covering part comprises detachable closing and/or partial means of the second end. In some of these embodiments, such detachable closing and/or partial means are advantageously formed by a towel rail (12). The towel rail is formed for example by a strip of elastic textile fixed on the covering part such that the part between the two ends of the strip can be lifted so that a towel can be introduced. This towel recloses the second end of the covering part.

In some embodiments, the inner surface of the covering part is covered in material preventing the user from being in direct contact with the heating layer, such as for example textile.

In some embodiments, the surface of the support intended to be in contact with the user is covered in material which prevents the user from being in direct contact with the support and any heating layer it can contain. Such material can be textile and damping material can be associated with it for the comfort of the patient. As the user breathes in the device, it is generally preferred that this material is impermeable or is detachably arranged on the surface.

In some embodiments, the covering part (1) comprises at least two portions whereof a first portion (1a) and a second portion (1b). The first portion (1a) can retract on or in the second portion (1b) by sliding of the first portion (1a) according to the longitudinal axis (11) of the covering part (1). So, for example, when the first portion (1a) retracts on the second portion (1b), the first portion (1a) covers the second portion (1b). It is preferred in general that the portion which covers the other is that which corresponds to the end near which the user will have his head, in this case the first portion (1a) in illustrative and non-limiting figures.

In some embodiments, the sliding is permitted by a sliding system (7). According to a particular configuration, such a sliding system is obtained by runners such as drawer mechanisms. In another configuration such a sliding system can comprise at least one slider fixed on the first portion (1a) of the covering part (1). The slider(s) are fixed parallel to the longitudinal axis (11) of the covering part (1) at the end of the branches of the parabolic form. The slider(s) are each capable of moving in a slide fixed on the support (2) according to a longitudinal axis (11) of the covering part (1). In another configuration the slider (7a) is fixed to the outer surface of the second portion (1b) parallel to the longitudinal axis (11) of the covering part (1) at the ends of the branches of the parabolic form. The slides (7b) in which the sliders (7a) can slide are fixed on the inner surface of the first portion (1a) parallel to the longitudinal axis (11) of the covering part (1) at the ends of the parabolic form.

In the illustrative and non-limiting configuration shown in FIG. 5, the covering part (1) comprises a first portion and a second portion. The first portion (1a) comprises an outer layer, two reflective layers (4) and two carbon plates (5a). The second portion (1b) also comprises an outer layer, two reflective layers (4) and two carbon plates (5a). The covering part is closed at an end by a rigid plate on which are fixed for example two carbon plates (5c). The support (2) also comprises at least one carbon plate.

In some embodiments, the carbon plates of the first portion are fed independently of the other plates.

In some embodiments, the carbon plates and/or the reflective layers are fixed against the outer layer by fastening means.

In some embodiments, the carbon plates of the second portion are fed independently of the other plates.

In some embodiments, the carbon plates of the support (2) are fed independently of the other plates.

In these embodiments where the plates are fed independently of each other, it is possible to modulate the infrared radiation according to the regions of the volume comprised between the covering part and the support (2). For example, the feed of the carbon plates of the first portion is regulated such that the infrared radiation produced has less intensity than the radiation produced by the carbon plates of the second portion.

In some embodiments, each portion and/or the support comprises several plates regulated independently of each other so as to provide even finer modulation of the infrared radiation, for example according to the areas to be treated in the case of phlebology.

In some embodiments, the device comprises regulating means for adjusting the intensity of radiation of the heating layers.

Independently of the form of the profile of the covering part, the irradiation device comprises a lifting system of the covering part. The first portion (1a) or the second portion (1b) of the covering part (1) is fixed on at least the lifting system of the covering part (1). The lifting system of the covering part (1) comprises at least one thrust jack (8) whereof an end is fixed on the support (2) or on the feet of the support (2) and the other end is fixed on the first or the second portion (1b) of the covering part (1) such that the jack (8) can exert thrust tending to lift an end of the covering part (1). The other end of the covering part (1) is fixed on the support (2) by means of a hinge allowing the longitudinal axis (11) of the covering part (1) to form a non-zero angle with the plane of the support (2). The thrust force of the jack(s) (8) is determined as a function of different positions of the first portion (1a). When the first portion (1a) does not cover the second portion (1b), the thrust force of the jack (8) is not sufficient to lift the non-fixed end of the covering part (1) whereof the longitudinal axis (11) remains parallel with the plane of the support (2). When the first portion (1a) covers the second portion (1b), the force of the jack (8) is sufficient to lift the fixed end of the covering part (1) whereof the longitudinal axis (11) forms a non-zero angle with the plane of the support (2). A stop element holds the covering part (1) when it is lifted by the jack(s) (8) to limit the angle formed by the longitudinal axis (11) of the covering part (1) and the plane of the support (2).

This arrangement has the advantage of easier opening of the device by the user himself not pushing on the first portion to make it slide and the first and the second portion lift up automatically under the action of the jack.

The invention therefore relates to any device comprising a support (2) and a covering part (1) arranged according to a longitudinal axis of the device. The covering part comprises a first portion slidably mounted on a second portion fixed by a pivoting axis on the support. The device comprises a jack mounted between the second portion and the support (2) or the feet of the support (2), the thrust force of the jack being adapted so that on the one hand the weight of the portions in deployed position prevents lifting of the two portions, and on the other hand when the portions are retracted at least partially, the force of the jack suffices to automatically lift the covering part.

The present description details different embodiments and configurations or variants in reference to figures and/or technical characteristics. Those skilled in the art will understand that the various technical characteristics of the various modes or configurations can be combined together to create other embodiments and configurations, unless specified otherwise or these technical characteristics are incompatible. Similarly, a technical characteristic of an embodiment or a configuration can be isolated from the other technical characteristics of this embodiment unless specified otherwise. In the present description, many specific details are provided by way of illustration and non-limiting so as to precisely detail the invention. Those skilled in the art, however, will understand that the invention can be executed in the absence of one or more of these specific details or with variants. On other occasions, some aspects are not detailed so as to prevent obscuring and weighing down the present description and those skilled in the art will understand various and varied means could be used and the invention is not limited to only those examples described.

It must be evident for those skilled in the art that the present invention allows embodiments in many other specific forms without departing from the field of application of the invention as claimed. Consequently, the present embodiments must be considered by way of illustration, but can be modified in the field defined by the scope of the appended claims, and the invention must not be limited to the details given hereinabove.

1. An infrared irradiation device comprising a longitudinal axis parallel to which are arranged a support capable of receiving a user and a covering part capable of covering the user, the covering part comprising at least one heating layer capable of emitting infrared radiation in at least one part of a volume located between an inner face of the at least one heating layer and the support, wherein the at least one heating layer comprises at least one carbon plate for emission of infrared radiation, a profile of the at least one heating layer, defined as the section transversal to the longitudinal axis, follows a form of parabolic curve.

2. The device according to claim 1, wherein an equation of the parabolic curve is y=x²−x−1.

3. The device according to claim 1, wherein the covering part further comprises a reflective layer of infrared radiation.

4. The device according to claim 3, wherein the reflective layer follows the profile of the at least one heating layer on an outer face of the heating layer.

5. The device according to claim 1, wherein the support comprises a second heating layer capable of emitting radiation in at least one part of the volume between the inner face of the first heating layer and the support.

6. The device according to claim 1, wherein an end, according to the longitudinal axis, of the covering part comprises a third heating layer closing the covering part.

7. The device according to claim 1, wherein the covering part comprises at least two portions of parabolic profiles, a second portion of the covering part being capable of sliding relative to a first portion parallel to the longitudinal axis so as to retract in or on the first portion.

8. The device according to claim 7, wherein the first portion is secured to at least one slide capable of guiding a slider.

9. The device according to claim 7, wherein the second portion is secured to at least one slider on its inner face capable of sliding on at least the slide of the first portion.

10. The device according to claim 1, wherein the covering part comprises an outer layer protecting the interior of the covering part.

11. The device according to claim 10, wherein the outer layer has a form which follows the form of the heating layer.

12. (canceled)

13. The device according to claim 3, wherein the reflective layer comprises at least one layer made of aluminum.

14. The device according to claim 10, wherein the outer layer is made of wood.

15. The device according to claim 1, wherein the support is made of wood.

16. The device according to claim 1, the wherein an inner surface of the covering part is covered in textile.

17. The device according to claim 1, wherein the irradiation device comprises a lifting system of the covering part, a first portion or a second portion of the covering part being fixed on at least the lifting system of the covering part, the lifting system of the covering part comprising at least one thrust jack whereof an end is fixed on the support or on the feet of the support and the other end is fixed on the first or the second portion of the covering part such that the jack can exert thrust tending to lift an end of the covering part, the other end of the covering part being fixed on the support by means of a hinge allowing the longitudinal axis of the covering part to form a non-zero angle with a plane of the support.
